(19) United States
(12) Patent Application Publication
(10) Pub. No.: US 2025/0252558 A1
(43) Pub. Date: Aug. 7, 2025

(54) ESTIMATING OPTIMAL RADIATION DOSE FOR X-RAY DIAGNOSTIC IMAGE

(71) Applicant: Fairchild Imaging Inc., San Jose, CA (US)

(72) Inventor: Glen COLLIER, Morgan Hill, CA (US)

(73) Assignee: Fairchild Imaging Inc., San Jose, CA (US)

(21) Appl. No.: 18/433,020

(22) Filed: Feb. 5, 2024

(51) Int. Cl. G06T 7/00; A61B 6/00

(52) U.S. Cl. CPC G06T 7/0012; A61B 6/542; G06T 2207/10116; G06T 2207/20081; G06T 2207/30168

(57) ABSTRACT

Techniques are provided for estimating an optimal radiation dose for x-ray imaging. A methodology implementing the techniques according to an embodiment includes operating an x-ray sensor at a first gain setting. The method also includes capturing a scouting image at the first gain setting using a scouting image radiation dose. The method further includes employing a radiation dose model to provide a diagnostic image radiation dose based on the scouting image. The diagnostic image radiation dose is greater than the scouting image radiation dose. The method further includes operating the x-ray sensor at a second gain setting. The second gain setting is less than the first gain setting. The method further includes capturing a diagnostic image using the provided diagnostic image radiation dose.

### FIELD OF DISCLOSURE

The present disclosure relates to x-ray imaging, and more particularly to estimating an optimal radiation dose for a diagnostic image.

### BACKGROUND

The selection of an appropriate radiation dose for x-ray imaging is typically a responsibility of the operator or radiographer. The dose selection is often estimated based on experience and is subject to error. Some studies have shown that 60 percent of images are improperly exposed, with 40 percent being unusable for diagnosis. When an image is over or under-exposed, the clinician (e.g., a doctor, dentist, or veterinarian, etc.) may make an incorrect diagnosis or may be unable to make any diagnosis. In such cases a follow-up image may be required, subjecting the patient to an undesirable additional dose of radiation.

### BRIEF DESCRIPTION OF THE DRAWINGS

FIG. 1 illustrates an x-ray system configured to provide optimal radiation dosing, in accordance with certain embodiments of the present disclosure.

FIG. 2 is a block diagram of the x-ray machine of FIG. 1, configured in accordance with certain embodiments of the present disclosure.

FIG. 3 is a block diagram illustrating operation of the controller of FIG. 1, configured in accordance with certain embodiments of the present disclosure.

FIG. 4 is a flowchart illustrating a methodology for training the radiation dose model of FIG. 1, in accordance with an embodiment of the present disclosure.

FIG. 5 is a flowchart illustrating a methodology for estimating an optimal radiation dose, in accordance with an embodiment of the present disclosure.

FIG. 6 is a block diagram of a processing platform configured to control the x-ray machine of FIG. 1, in accordance with an embodiment of the present disclosure.

Although the following Detailed Description will proceed with reference being made to illustrative embodiments, many alternatives, modifications, and variations thereof will be apparent in light of this disclosure.

### DETAILED DESCRIPTION

Techniques are provided herein for estimating an optimal radiation dose for a diagnostic x-ray image. As noted above, relying on an operator to set radiation doses based on experience can be an error prone process, resulting in over or under-exposed images. In such cases a follow-up image may be required, subjecting the patient to further radiation.

To this end, and in accordance with an embodiment of the present disclosure, a system for estimating an optimal radiation dose for diagnostic x-ray imaging is disclosed. The system uses an initial low dose exploratory image, also referred to herein as a scouting image, to determine an appropriate dosage for a subsequent higher dose diagnostic image. The scouting image is obtained at a relatively high amplifier gain setting which allows an image to be obtained at the low radiation dose. Although the scouting image is not suitable for diagnostic use, since the image quality is reduced due to quantization and system noise that are amplified by the high gain, it can be used to determine an appropriate subsequent dose for obtaining a diagnostic image. In some embodiments, a machine learning algorithm or artificial intelligence (AI) based system is employed to estimate an optimal diagnostic image radiation dose based on the scouting image.

The disclosed system improves patient safety by reducing the possibility of generating improperly exposed diagnostic images that require retakes. The system also reduces the variability that results from operator estimation of radiation dosing. Additionally, the total dose radiation delivered to the patient, from the combination of scouting image and diagnostic image, may be less than the dose delivered by a conventional system where the diagnostic dose may be greater than necessary.

In accordance with an embodiment, a methodology includes controlling an x-ray sensor to operate at a first gain setting. The method also includes capturing a scouting image at the first gain setting using a scouting image radiation dose (e.g., a relatively low dose). The method further includes employing a radiation dose model to provide a diagnostic image radiation dose based on the scouting image. In some embodiments, the radiation dose model is a machine learning algorithm trained on a corpus or database of scouting images/diagnostic image pairing samples. In some embodiments, the diagnostic image radiation dose provided by the model is greater than the scouting image radiation dose. The method further includes controlling the x-ray sensor to operate at a second (e.g., lower) gain setting. The method further includes capturing a diagnostic image using the provided diagnostic image radiation dose at the second gain setting.

The disclosed system may be used to improve imaging in medical and dental applications for human patients. In some embodiments, the system may also be used on animals in veterinary applications. It will be appreciated that the techniques described herein may provide improved x-ray imaging with reduced radiation exposure to the patient, compared to systems that rely on manual adjustment of radiation dosage. Numerous embodiments and applications will be apparent in light of this disclosure.

### System Architecture

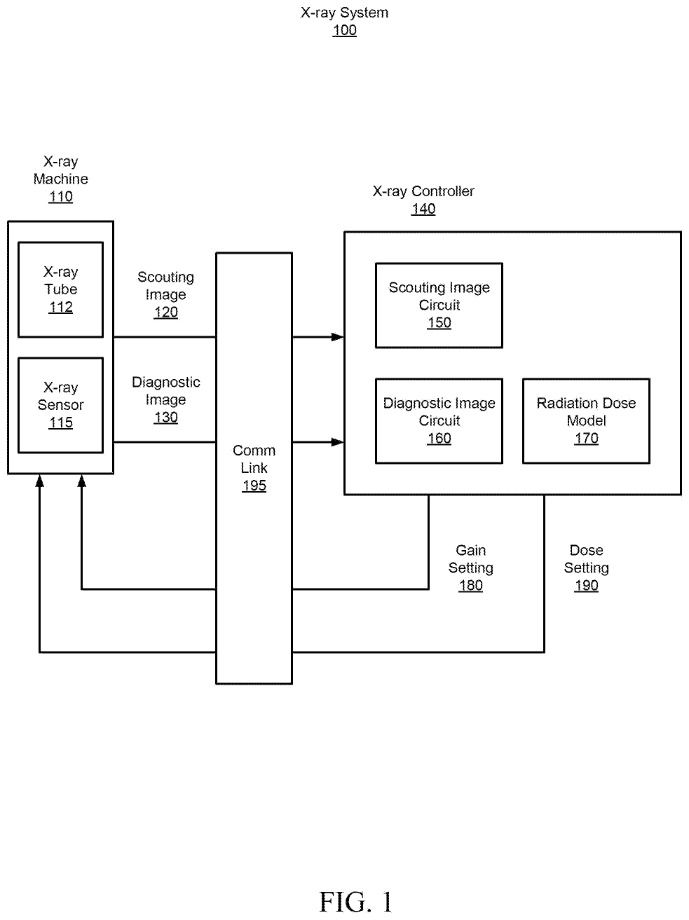

FIG. 1 illustrates an x-ray system 100 configured to provide improved imaging at an optimal radiation dosing, in accordance with certain embodiments of the present disclosure. The x-ray system 100 is shown to include an x-ray machine 110, a communications link 195, and an x-ray controller 140.

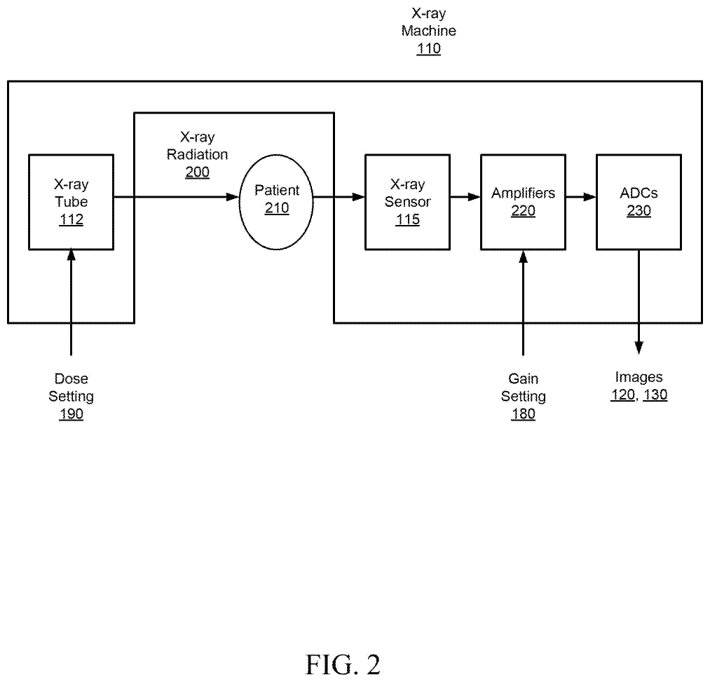

The x-ray machine 110 is configured to obtain x-ray images of any desired anatomical region of a patient, whether human or animal, including dental applications. The x-ray machine operates under the control of x-ray controller 140. Operation of the x-ray machine 110 will be described below in connection with FIG. 2, but at a high level, the x-ray machine is configured to generate and transmit x-ray radiation, for example using x-ray tube 112. The radiation passes through the subject to be imaged and is received by the x-ray sensor 115 from which images are generated.

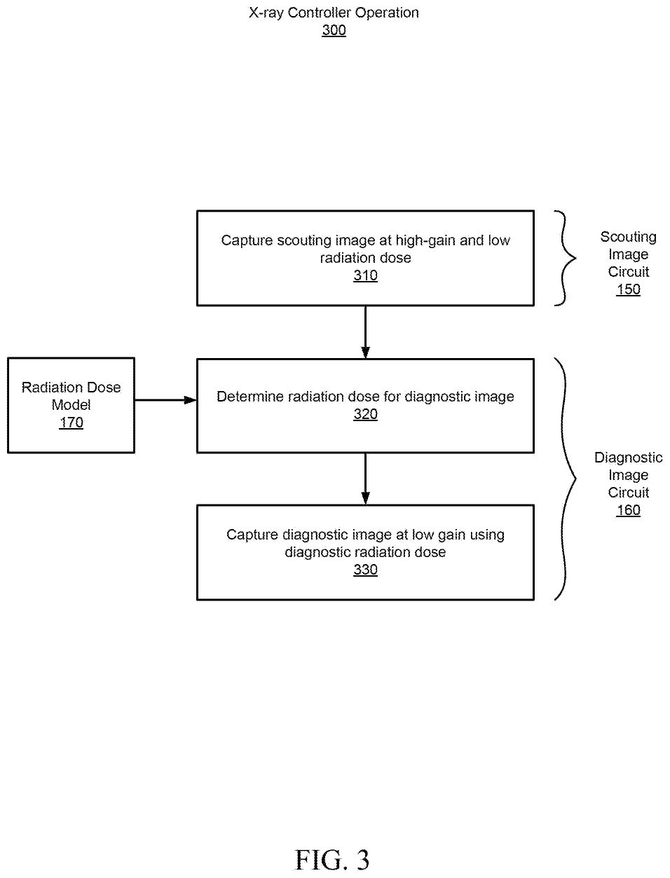

Operation of the x-ray controller 140 is described in greater detail below in connection with FIG. 3, but at a high level, the x-ray controller is configured to control the x-ray machine 110, for example by setting the gain values 180 and radiation doses 190 for the scouting image 120 and the diagnostic image 130. In some embodiments, control signals 180, 190 and images 120, 130 are transmitted and received through a communications link 195. In some embodiments, the communications link 195 may be a universal serial bus (USB), a Wireless Fidelity (Wi-Fi) link, or any other suitable communications link.

FIG. 2 is a block diagram of the x-ray machine 110 of FIG. 1, configured in accordance with certain embodiments of the present disclosure. The x-ray machine 110 is configured to obtain x-ray images of any desired anatomical region of a patient, whether human or animal, including images for dental applications. The x-ray tube (or emitter) 112 is configured to generate x-ray radiation 200 at a selected radiation dose setting 190, for example by controlling voltage and current levels through the tube. Any suitable x-ray emitter may be used. The x-ray radiation 200 pass through the patient or subject matter to be imaged and is detected by sensor pixels of the x-ray sensor 115. Analog electrical signals provided by the sensor pixels are amplified by amplifier 220 and fed to analog to digital converters (ADCs) 230 to generate digital images 120, 130. The amplification gain may be adjusted to any suitable value through gain setting 180. Generally, a lower radiation dose requires a higher amplification gain to produce an image, but the higher gain increases quantization and system noise, which reduces image quality.

FIG. 3 is a block diagram illustrating operation of the controller 140 of FIG. 1, configured in accordance with certain embodiments of the present disclosure. The x-ray controller 140 is shown to include the scouting image circuit 150, the diagnostic image circuit 160, and the radiation dose model 170. The scouting image circuit 150 and the diagnostic image circuit 160 are shown as separate circuits but may be implemented together in a common image circuit that has configurable settings, including settings for gain and radiation dose. In some examples, the scouting image circuit 150 and the diagnostic image circuit 160 each or collectively comprise a gain control circuit configured to set the gain of x-ray sensor 115 and a dosing control circuit configured to set the radiation dose of x-ray tube 112. Alternatively, or in addition to, the scouting image circuit 150 and the diagnostic image circuit 160 may comprise one or more processors and one or more memories encoded with instructions that when executed cause functionality of the scouting image circuit 150 and the diagnostic image circuit 160 to be carried out. In some embodiments, the x-ray controller 140 may be part of a host computer such as a desktop computer, workstation, laptop computer, or tablet computer which may include a display unit upon which images may be viewed by the operator.

The scouting image circuit 150 is configured to cause the x-ray sensor to operate at a first gain setting (e.g., the high gain setting) to capture a scouting image 120 using a scouting image radiation dose, as shown at operation 310. The scouting image circuit 150 transmits the gain setting 180 and dose setting 190 to the x-ray machine 110 and commands the x-ray machine to capture the scouting image 120. The scouting image circuit 150 retrieves the captured scouting image 120 through the communications link 195. In some examples, a graphical user interface (GUI) executing on the host computer may be used to receive the initial or exploratory gain setting 180 and dose setting 190 from the radiation dose model 170 and present those settings to an operator, so the operator can adjust the settings of the x-ray machine 110 accordingly (e.g., via GUI control feature or a physical control knob of the scouting image circuit 150). In other examples, such initial settings may be hard-coded into the scouting image circuit 150, or provided to the scouting image circuit 150 via the radiation dose model 170. In still other examples, such initial settings may be programmed into the scouting image circuit 150 at set-up time of the x-ray machine 110, or at the beginning of a patient x-ray session.

The radiation dose model 170 is configured to provide a diagnostic image radiation dose based on the scouting image, as shown at operation 320. The diagnostic image radiation dose is typically greater than the scouting image radiation dose. As described above, the radiation dose model 170 may also provide the initial gain setting and scouting image radiation dose to the scouting image circuit 150.

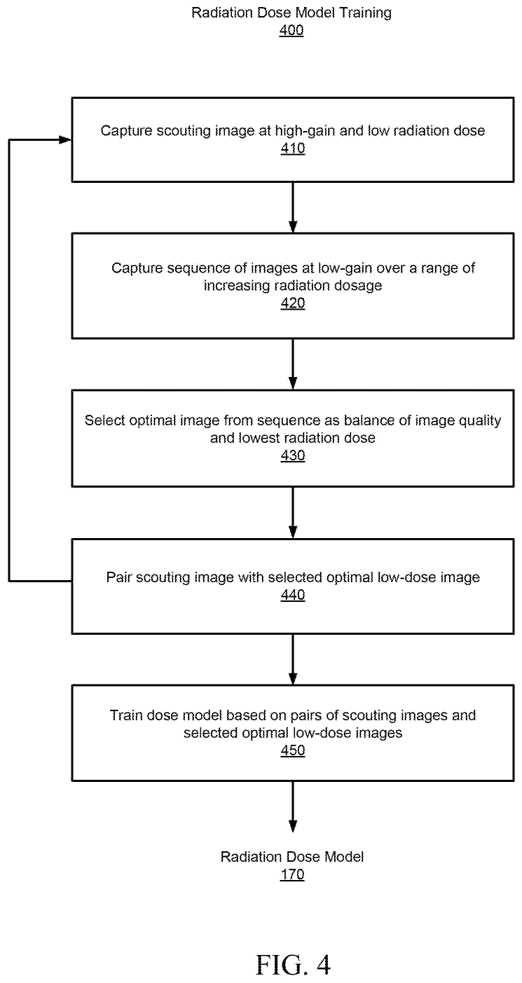

In some embodiments, the radiation dose model 170 is a machine learning algorithm trained on a corpus or database of pairings of sample scouting images and diagnostic images, along with the radiation dose used to achieve those diagnostic images, where the training diagnostic images are selected to provide a desired diagnostic image quality. A training process is described in greater detail below in connection with FIG. 4.

In some embodiments, the radiation dose model 170 is a mathematical algorithm configured to map scouting images to diagnostic images of usable quality based on an analysis of sample scouting images paired with sample diagnostic images of a desired diagnostic image quality along with the radiation dose used to achieve those diagnostic images.

The diagnostic image circuit 160 is configured to cause the x-ray sensor to operate at a second gain setting (e.g., the low gain setting) to capture a diagnostic image 130 using the diagnostic image radiation dose provided by the radiation dose model 170, as shown at operation 330. The diagnostic image circuit 150 transmits the gain setting 180 and dose setting 190 to the x-ray machine 110 and commands the x-ray machine to capture the diagnostic image 130. The diagnostic image circuit 160 retrieves the captured diagnostic image 130 through the communications link 195. The diagnostic image may then be displayed to the operator or saved for further use.

### Methodology

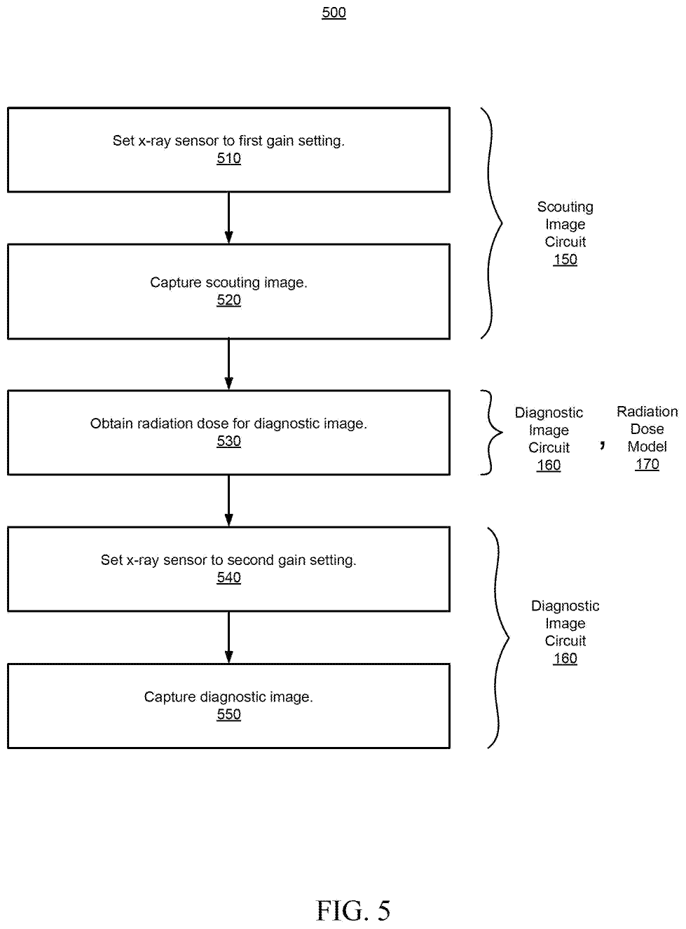

FIG. 4 is a flowchart illustrating a methodology 400 for training the radiation dose model 170 of FIG. 1 and FIG. 5 is a flowchart illustrating a methodology 500 for estimating an optimal diagnostic image radiation dose, in accordance with an embodiment of the present disclosure.

As can be seen, example methods 400 and 500 include a number of phases and sub-processes, the sequence of which may vary from one embodiment to another. However, when considered in aggregate, these phases and sub-processes form a process for operation of an x-ray system 100, in accordance with certain of the embodiments disclosed herein, for example as illustrated in FIGS. 1-3, as described above. However other system architectures can be used in other embodiments, as will be apparent in light of this disclosure. To this end, the correlation of the various functions shown in FIGS. 4 and 5 to the specific components illustrated in the figures, is not intended to imply any structural and/or use limitations. Rather other embodiments may include, for example, varying degrees of integration wherein multiple functionalities are effectively performed by one system. Numerous variations and alternative configurations will be apparent in light of this disclosure.

In one embodiment, the radiation dose model 170 is a machine learning algorithm and method 400 is used to train the radiation dose model. The training process includes capturing multiple x-ray images, as described below, which may be accomplished using commercially available anatomical samples to avoid harm to humans or animals from radiation exposure.

Training commences at operation 410, by setting the x-ray sensor to a relatively high gain mode and capturing a training scouting image at a relatively low radiation dose.

At operation 420, the x-ray sensor is set to a relatively low gain mode and a sequence of sample diagnostic images are captured over a series of radiation doses ranging from a relatively low dose to a relatively high dose.

At operation 430, an optimal diagnostic image is selected from the sequence of sample diagnostic images to be used as a training diagnostic image. The optimal diagnostic image is selected to provide a desired level of quality in the diagnostic image at the lowest radiation dosage. In some embodiments, a radiologist or other subject matter expert may employ their experience to select an optimal diagnostic image from the sequence by achieving a balance between providing one of the best images in the sequence and using the lowest radiation dose possible (or otherwise within a given threshold of that desired dose, such as within 5%, or better).

At operation 440, the training scouting image is paired with the selected training diagnostic image, along with a label indicating the radiation dose used to achieve that diagnostic image, to form a training image pair which is added to a collection of training image pairs. The process may then iterate back to operation 410 to obtain additional training image pairs and radiation doses to build a labeled training data set. A given training image pair may also be labeled as ideal or otherwise marked as an example of a desired result, and a number of such training image pairs within an acceptable tolerance of a target (e.g., within +/−5%, or better to provide a symmetric tolerance, or within +2% and −7% to provide an asymmetric tolerance that favors lower dosing over higher dosing) may be so labelled or otherwise marked as an acceptable standard. In this manner, there may be more than one optimal diagnostic image (rather than just the absolute best one, for a given data set). In some examples, additional training image pairs that represent results outside of the desired threshold or range may also be generated, with such training image pairs being labelled as non-ideal and with a corresponding radiation dose, to further improve the labeled training data set.

At operation 450, when a sufficient number of training image pairs have been generated, the radiation dose model is trained on the labeled training data set to generate radiation dose model 170.

In some embodiments, the radiation dose model may be an analytical mathematical algorithm, as an alternative to the machine learning algorithm. The analytical mathematical algorithm may be designed, for example by a subject matter expert experienced in the field of imaging. The algorithm design may be based on an analysis of a collection of sample scouting images paired with sample diagnostic images and associated diagnostic radiation doses. The algorithm may include formulas designed to map scouting images to diagnostic images of usable quality, along with the radiation dose used to achieve those diagnostic images. The algorithm may also include formulas designed to map scouting images to diagnostic images of unusable quality, along with the radiation dose used to achieve those diagnostic images. Again, ranging may be used to allow for multiple possible results that are usable or otherwise acceptable, rather than just a specific optimal result.

Method 500, for estimating an optimal diagnostic image radiation dose, commences, at operation 510 by controlling the x-ray sensor to operate at a first gain setting (e.g., a relatively high gain setting).

At operation 520, a scouting image is captured by the x-ray sensor at the high gain setting using a scouting image radiation dose (e.g., a relatively low radiation dose).

At operation 530, the radiation dose model is employed to provide a diagnostic image radiation dose based on the scouting image. This identified diagnostic image radiation dose may be associated with a singular image paired with the scouting image or may be associated with one of a set of images that each would be acceptable. In the latter case, an operator may be given an opportunity to select from the set of acceptable diagnostic images paired with the scouting image, such as via a graphical user interface that provides the diagnostic images and corresponding doses. Such presentation may be helpful in training the human operator as well. In other examples, no human interaction is needed in the selection process.

At operation 540, the x-ray sensor is controlled to operate at a second gain setting (e.g., a relatively low gain setting that is less than the high gain setting).

At operation 550, a diagnostic image is captured by the x-ray sensor at the low gain setting using the provided diagnostic image radiation dose, which is greater than the scouting image radiation dose.

In some embodiments, the x-ray controller issues commands to the x-ray sensor over a communications link to control the gain settings and radiation dose. The communications link may also be used to obtain the captured scouting image and diagnostic image from the x-ray sensor.

### Example System

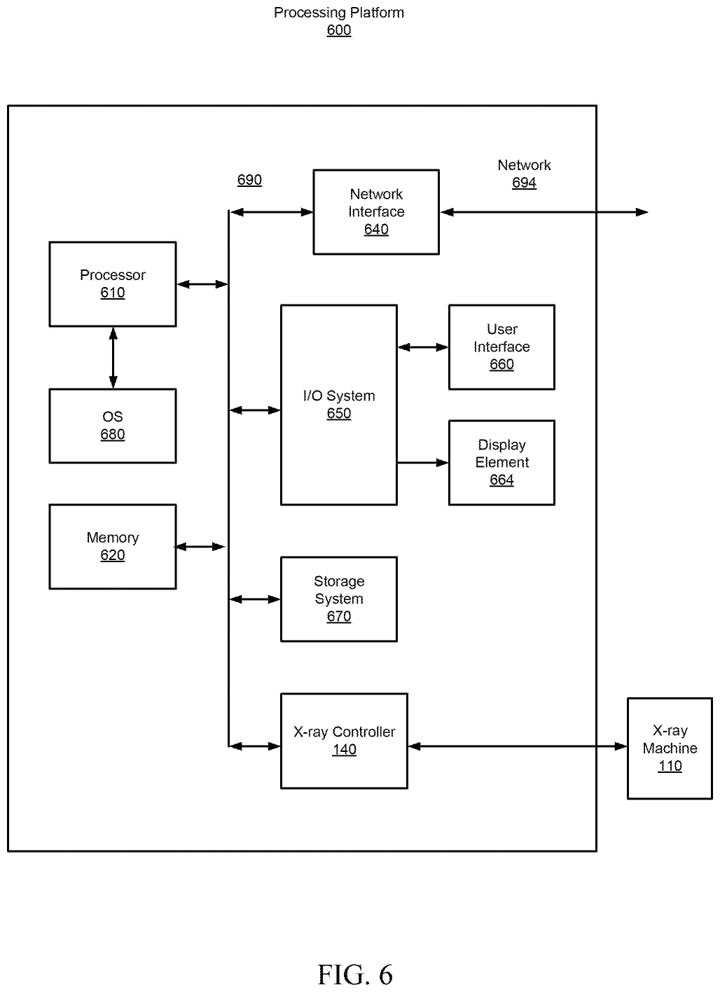

FIG. 6 is a block diagram of a processing platform 600 configured to control the x-ray machine 110 of FIG. 1, in accordance with an embodiment of the present disclosure. In some embodiments, platform 600, or portions thereof, may be hosted on, or otherwise be incorporated into a desktop computer, workstation, laptop computer, or tablet computer. The disclosed techniques may be used to provide an optimal x-ray dose for diagnostic imaging.

In some embodiments, platform 600 may comprise any combination of a processor 610, memory 620, x-ray controller 140, a network interface 640, an input/output (I/O) system 650, a user interface 660, a display element 664, and a storage system 670. As can be further seen, a bus and/or interconnect 690 is also provided to allow for communication between the various components listed above and/or other components not shown. Platform 600 can be coupled to a network 694 through network interface 640 to allow for communications with other computing devices, platforms, devices to be controlled, or other resources. Other componentry and functionality not reflected in the block diagram of FIG. 6 will be apparent in light of this disclosure, and it will be appreciated that other embodiments are not limited to any particular hardware configuration.

Processor 610 can be any suitable processor, and may include one or more coprocessors or controllers, such as an audio processor, a graphics processing unit, or hardware accelerator, to assist in control and processing operations associated with platform 600, including operation of the x-ray controller 140. In some embodiments, the processor 610 may be implemented as any number of processor cores. The processor (or processor cores) may be any type of processor, such as, for example, a micro-processor, an embedded processor, a digital signal processor (DSP), a graphics processor (GPU), a tensor processing unit (TPU), a network processor, a field programmable gate array or other device configured to execute code. The processors may be multithreaded cores in that they may include more than one hardware thread context (or “logical processor”) per core. Processor 610 may be implemented as a complex instruction set computer (CISC) or a reduced instruction set computer (RISC) processor. In some embodiments, processor 610 may be configured as an x86 instruction set compatible processor.

Memory 620 can be implemented using any suitable type of digital storage including, for example, flash memory and/or random access memory (RAM). In some embodiments, the memory 620 may include various layers of memory hierarchy and/or memory caches as are known to those of skill in the art. Memory 620 may be implemented as a volatile memory device such as, but not limited to, a RAM, dynamic RAM (DRAM), or static RAM (SRAM) device. Storage system 670 may be implemented as a non-volatile storage device such as, but not limited to, one or more of a hard disk drive (HDD), a solid-state drive (SSD), a universal serial bus (USB) drive, an optical disk drive, tape drive, an internal storage device, an attached storage device, flash memory, battery backed-up synchronous DRAM (SDRAM), and/or a network accessible storage device.

Processor 610 may be configured to execute an Operating System (OS) 680 which may comprise any suitable operating system, such as Google Android (Google Inc., Mountain View, CA), Microsoft Windows (Microsoft Corp., Redmond, WA), Apple OS X (Apple Inc., Cupertino, CA), Linux, or a real-time operating system (RTOS). As will be appreciated in light of this disclosure, the techniques provided herein can be implemented without regard to the particular operating system provided in conjunction with platform 600, and therefore may also be implemented using any suitable existing or subsequently-developed platform.

Network interface circuit 640 can be any appropriate network chip or chipset which allows for wired and/or wireless connection between other components of platform 600 and/or network 694, thereby enabling platform 600 to communicate with other local and/or remote computing systems, and/or other resources. Wired communication may conform to existing (or yet to be developed) standards, such as, for example, Ethernet. Wireless communication may conform to existing (or yet to be developed) standards, such as, for example, cellular communications including LTE (Long Term Evolution) and 5G, Wireless Fidelity (Wi-Fi), Bluetooth, and/or Near Field Communication (NFC). Exemplary wireless networks include, but are not limited to, wireless local area networks, wireless personal area networks, wireless metropolitan area networks, cellular networks, and satellite networks.

I/O system 650 may be configured to interface between various I/O devices and other components of platform 600. I/O devices may include, but not be limited to, user interface 660 and display element 664. User interface 660 may include devices (not shown) such as a touchpad, keyboard, and mouse, etc., for example, to allow the user to control the system. Display element 664 may be configured to display diagnostic images and other suitable information to a clinician or operator of the system. I/O system 650 may include a graphics subsystem configured to perform processing of images for rendering on the display element 664. Graphics subsystem may be a graphics processing unit or a visual processing unit (VPU), for example. An analog or digital interface may be used to communicatively couple graphics subsystem and the display element. For example, the interface may be any of a high definition multimedia interface (HDMI), DisplayPort, wireless HDMI, and/or any other suitable interface using wireless high definition compliant techniques. In some embodiments, the graphics subsystem could be integrated into processor 610 or any chipset of platform 600.

It will be appreciated that in some embodiments, the various components of platform 600 may be combined or integrated in a system-on-a-chip (SoC) architecture. In some embodiments, the components may be hardware components, firmware components, software components or any suitable combination of hardware, firmware, or software.

X-ray controller 140 is configured to estimate and employ an optimal x-ray radiation dose for diagnostic imaging, as described above. X-ray controller 140 may include any or all of the circuits/components illustrated in FIGS. 1-3, as described above. These components can be implemented or otherwise used in conjunction with a variety of suitable software and/or hardware that is coupled to or that otherwise forms a part of platform 600. These components can additionally or alternatively be implemented or otherwise used in conjunction with user I/O devices that are capable of providing information to, and receiving information and commands from, a user.

In various embodiments, platform 600 may be implemented as a wireless system, a wired system, or a combination of both. When implemented as a wireless system, platform 600 may include components and interfaces suitable for communicating over a wireless shared media, such as one or more antennae, transmitters, receivers, transceivers, amplifiers, filters, control logic, and so forth. An example of wireless shared media may include portions of a wireless spectrum, such as the radio frequency spectrum and so forth. When implemented as a wired system, platform 600 may include components and interfaces suitable for communicating over wired communications media, such as input/output adapters, physical connectors to connect the input/output adaptor with a corresponding wired communications medium, a network interface card (NIC), disc controller, video controller, audio controller, and so forth. Examples of wired communications media may include a wire, cable metal leads, printed circuit board (PCB), backplane, switch fabric, semiconductor material, twisted pair wire, coaxial cable, fiber optics, and so forth.

Various embodiments may be implemented using hardware elements, software elements, or a combination of both. Examples of hardware elements may include processors, microprocessors, circuits, circuit elements (for example, transistors, resistors, capacitors, inductors, and so forth), integrated circuits, ASICs, programmable logic devices, digital signal processors, FPGAs, logic gates, registers, semiconductor devices, chips, microchips, chipsets, and so forth. Examples of software may include software components, programs, applications, computer programs, application programs, system programs, machine programs, operating system software, middleware, firmware, software modules, routines, subroutines, functions, methods, procedures, software interfaces, application program interfaces, instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power level, heat tolerances, processing cycle budget, input data rates, output data rates, memory resources, data bus speeds, and other design or performance constraints.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still cooperate or interact with each other.

The various embodiments disclosed herein can be implemented in various forms of hardware, software, firmware, and/or special purpose processors. For example, in one embodiment at least one non-transitory computer readable storage medium has instructions encoded thereon that, when executed by one or more processors, cause one or more of the methodologies disclosed herein to be implemented. The instructions can be encoded using a suitable programming language, such as C, C++, object oriented C, Java, JavaScript, Visual Basic .NET, Beginner's All-Purpose Symbolic Instruction Code (BASIC), or alternatively, using custom or proprietary instruction sets. The instructions can be provided in the form of one or more computer software applications and/or applets that are tangibly embodied on a memory device, and that can be executed by a computer having any suitable architecture. In one embodiment, the system can be hosted on a given website and implemented, for example, using JavaScript or another suitable browser-based technology. For instance, in certain embodiments, the system may leverage processing resources provided by a remote computer system accessible via network 694. The computer software applications disclosed herein may include any number of different modules, sub-modules, or other components of distinct functionality, and can provide information to, or receive information from, still other components. These modules can be used, for example, to communicate with input and/or output devices such as a display screen, a touch sensitive surface, a printer, and/or any other suitable device. Other componentry and functionality not reflected in the illustrations will be apparent in light of this disclosure, and it will be appreciated that other embodiments are not limited to any particular hardware or software configuration. Thus, in other embodiments platform 600 may comprise additional, fewer, or alternative subcomponents as compared to those included in the example embodiment of FIG. 6.

The aforementioned non-transitory computer readable medium may be any suitable medium for storing digital information, such as a hard drive, a server, a flash memory, and/or random-access memory (RAM), or a combination of memories. In alternative embodiments, the components and/or modules disclosed herein can be implemented with hardware, including gate level logic such as a field-programmable gate array (FPGA), or alternatively, a purpose-built semiconductor such as an application-specific integrated circuit (ASIC). Still other embodiments may be implemented with a microcontroller having a number of input/output ports for receiving and outputting data, and a number of embedded routines for carrying out the various functionalities disclosed herein. It will be apparent that any suitable combination of hardware, software, and firmware can be used, and that other embodiments are not limited to any particular system architecture.

Some embodiments may be implemented, for example, using a machine readable medium or article which may store an instruction or a set of instructions that, if executed by a machine, may cause the machine to perform a method, process, and/or operations in accordance with the embodiments. Such a machine may include, for example, any suitable processing platform, computing platform, computing device, processing device, computing system, processing system, computer, process, or the like, and may be implemented using any suitable combination of hardware and/or software. The machine readable medium or article may include, for example, any suitable type of memory unit, memory device, memory article, memory medium, storage device, storage article, storage medium, and/or storage unit, such as memory, removable or non-removable media, erasable or non-erasable media, writeable or rewriteable media, digital or analog media, hard disk, floppy disk, compact disk read only memory (CD-ROM), compact disk recordable (CD-R) memory, compact disk rewriteable (CD-RW) memory, optical disk, magnetic media, magneto-optical media, removable memory cards or disks, various types of digital versatile disk (DVD), a tape, a cassette, or the like. The instructions may include any suitable type of code, such as source code, compiled code, interpreted code, executable code, static code, dynamic code, encrypted code, and the like, implemented using any suitable high level, low level, object oriented, visual, compiled, and/or interpreted programming language.

Unless specifically stated otherwise, it may be appreciated that terms such as “processing,” “computing,” “calculating,” “determining,” or the like refer to the action and/or process of a computer or computing system, or similar electronic computing device, that manipulates and/or transforms data represented as physical quantities (for example, electronic) within the registers and/or memory units of the computer system into other data similarly represented as physical entities within the registers, memory units, or other such information storage transmission or displays of the computer system. The embodiments are not limited in this context.

The terms “circuit” or “circuitry,” as used in any embodiment herein, are functional and may comprise, for example, singly or in any combination, hardwired circuitry, programmable circuitry such as computer processors comprising one or more individual instruction processing cores, state machine circuitry, and/or firmware that stores instructions executed by programmable circuitry. The circuitry may include a processor and/or controller configured to execute one or more instructions to perform one or more operations described herein. The instructions may be embodied as, for example, an application, software, firmware, etc. configured to cause the circuitry to perform any of the aforementioned operations. Software may be embodied as a software package, code, instructions, instruction sets and/or data recorded on a computer-readable storage device. Software may be embodied or implemented to include any number of processes, and processes, in turn, may be embodied or implemented to include any number of threads, etc., in a hierarchical fashion. Firmware may be embodied as code, instructions or instruction sets and/or data that are hard-coded (e.g., nonvolatile) in memory devices. The circuitry may, collectively or individually, be embodied as circuitry that forms part of a larger system, for example, an integrated circuit (IC), an application-specific integrated circuit (ASIC), a system-on-a-chip (SoC), desktop computers, laptop computers, tablet computers, servers, smartphones, etc. Other embodiments may be implemented as software executed by a programmable control device. In such cases, the terms “circuit” or “circuitry” are intended to include a combination of software and hardware such as a programmable control device or a processor capable of executing the software. As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Numerous specific details have been set forth herein to provide a thorough understanding of the embodiments. It will be understood, however, that other embodiments may be practiced without these specific details, or otherwise with a different set of details. It will be further appreciated that the specific structural and functional details disclosed herein are representative of example embodiments and are not necessarily intended to limit the scope of the present disclosure. In addition, although the subject matter has been described in language specific to structural features and/or methodological acts, it is to be understood that the subject matter defined in the appended claims is not necessarily limited to the specific features or acts described herein. Rather, the specific features and acts described herein are disclosed as example forms of implementing the claims.

### FURTHER EXAMPLE EMBODIMENTS

The following examples pertain to further embodiments, from which numerous permutations and configurations will be apparent.

Example 1 is a system for estimating an optimal radiation dose comprising: a scouting image circuit configured to cause an x-ray sensor to operate at a first gain setting to capture a scouting image using a scouting image radiation dose; a radiation dose model configured to provide a diagnostic image radiation dose based on the scouting image; and a diagnostic image circuit configured to cause the x-ray sensor to operate at a second gain setting to capture a diagnostic image using the provided diagnostic image radiation dose, wherein the second gain setting is less than the first gain setting.

Example 2 includes the system of Example 1, wherein the diagnostic image radiation dose is greater than the scouting image radiation dose.

Example 3 includes the system of Examples 1 or 2, wherein the radiation dose model is a machine learning algorithm trained on a collection of training scouting images paired with training diagnostic images, the training diagnostic images selected to provide a desired diagnostic image quality.

Example 4 includes the system of any of Examples 1-3, wherein the radiation dose model is an algorithm, the algorithm configured based on an analysis of a collection of sample scouting images paired with sample diagnostic images, the sample diagnostic images selected to provide a desired diagnostic image quality.

Example 5 includes the system of any of Examples 1-4, further comprising a communications link between a controller and the x-ray sensor, the controller comprising the scouting image circuit and the diagnostic image circuit, wherein the communications link is configured to provide gain settings to the x-ray sensor and radiation dose settings to an x-ray emitter, and to receive the scouting image and the diagnostic image from the x-ray sensor.

Example 7 includes the system of Example 6, wherein the scouting image circuit and diagnostic image circuit comprise a gain control circuit configured to set gain of the x-ray sensor and a dosing control circuit configured to set radiation dose of an x-ray emitter.

Example 8 includes the system of any of Examples 1-7, wherein the scouting image circuit and the diagnostic image circuit comprise one or more processors and one or more memories encoded with instructions.

Example 9 is an x-ray system including the system of any of Examples 1-8.

Example 10 is a computer program product including one or more non-transitory machine-readable mediums encoded with instructions that when executed by one or more processors cause a process to be carried out for estimating an optimal radiation dose, the process comprising: operating an x-ray sensor at a first gain setting; capturing a scouting image at the first gain setting using a scouting image radiation dose; employing a radiation dose model to provide a diagnostic image radiation dose based on the scouting image; operating the x-ray sensor at a second gain setting, the second gain setting less than the first gain setting; and capturing a diagnostic image using the provided diagnostic image radiation dose.

Example 11 includes the computer program product of Example 10, wherein the diagnostic image radiation dose is greater than the scouting image radiation dose.

Example 12 includes the computer program product of Examples 10 or 11, wherein the radiation dose model is a machine learning algorithm trained on a collection of training scouting images paired with training diagnostic images, the training diagnostic images selected to provide a desired diagnostic image quality.

Example 13 includes the computer program product of any of Examples 10-12, wherein the radiation dose model is an algorithm, the algorithm configured based on an analysis of a collection of sample scouting images paired with sample diagnostic images, the sample diagnostic images selected to provide a desired diagnostic image quality.

Example 14 includes the computer program product of any of Examples 10-13, wherein the process further comprises communicating gain settings and radiation dose settings to the x-ray sensor over a communications link and receiving the scouting image and the diagnostic image from the x-ray sensor over a communications link.

Example 15 is an x-ray system comprising the computer program product of any of Examples 10-14.

Example 16 is a method for estimating an optimal radiation dose, the method comprising: operating, by a processor-based system, an x-ray sensor at a first gain setting; capturing, by the processor-based system, a scouting image at the first gain setting using a scouting image radiation dose; employing, by the processor-based system, a radiation dose model to provide a diagnostic image radiation dose based on the scouting image; operating, by the processor-based system, the x-ray sensor at a second gain setting, the second gain setting less than the first gain setting; and capturing, by the processor-based system, a diagnostic image using the provided diagnostic image radiation dose.

Example 17 includes the method of Example 16, wherein the diagnostic image radiation dose is greater than the scouting image radiation dose.

Example 18 includes the method of Examples 16 or 17, wherein the radiation dose model is a machine learning algorithm trained on a collection of training scouting images paired with training diagnostic images, the training diagnostic images selected to provide a desired diagnostic image quality.

Example 19 includes the method of any of Examples 16-18, wherein the radiation dose model is an algorithm, the algorithm configured based on an analysis of a collection of sample scouting images paired with sample diagnostic images, the sample diagnostic images selected to provide a desired diagnostic image quality.

Example 20 includes the method of any of Examples 16-19, further comprising communicating gain settings to the x-ray sensor and radiation dose settings to an x-ray emitter over a communications link and receiving the scouting image and the diagnostic image from the x-ray sensor over a communications link.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be appreciated in light of this disclosure. The present disclosure should, therefore, be considered to encompass such combinations, variations, and modifications. It is intended that the scope of the present disclosure be limited not by this detailed description, but rather by the claims appended hereto. Future filed applications claiming priority to this application may claim the disclosed subject matter in a different manner and may generally include any set of one or more elements as variously disclosed or otherwise demonstrated herein.

What is claimed is:

1. A system for estimating an optimal radiation dose comprising:
a scouting image circuit configured to cause an x-ray sensor to operate at a first gain setting to capture a scouting image using a scouting image radiation dose;
a radiation dose model configured to provide a diagnostic image radiation dose based on the scouting image; and
a diagnostic image circuit configured to cause the x-ray sensor to operate at a second gain setting to capture a diagnostic image using the provided diagnostic image radiation dose, wherein the second gain setting is less than the first gain setting.

2. The system of claim 1, wherein the diagnostic image radiation dose is greater than the scouting image radiation dose.

3. The system of claim 1, wherein the radiation dose model is a machine learning algorithm trained on a collection of training scouting images paired with training diagnostic images, the training diagnostic images selected to provide a desired diagnostic image quality.

4. The system of claim 1, wherein the radiation dose model is an algorithm, the algorithm configured based on an analysis of a collection of sample scouting images paired with sample diagnostic images, the sample diagnostic images selected to provide a desired diagnostic image quality.

5. The system of claim 1, further comprising a communications link between a controller and the x-ray sensor, the controller comprising the scouting image circuit and the diagnostic image circuit, wherein the communications link is configured to provide gain settings to the x-ray sensor and radiation dose settings to an x-ray emitter, and to receive the scouting image and the diagnostic image from the x-ray sensor.

6. The system of claim 5, wherein the scouting image circuit and diagnostic image circuit comprise a gain control circuit configured to set gain of the x-ray sensor.

7. The system of claim 5, wherein the scouting image circuit and diagnostic image circuit comprise a dosing control circuit configured to set radiation dose of an x-ray emitter.

8. The system of claim 1, wherein the scouting image circuit and the diagnostic image circuit comprise one or more processors and one or more memories encoded with instructions.

9. An x-ray system comprising the system of claim 1.

10. A computer program product including one or more non-transitory machine-readable mediums encoded with instructions that when executed by one or more processors cause a process to be carried out for estimating an optimal radiation dose, the process comprising:
operating an x-ray sensor at a first gain setting;
capturing a scouting image at the first gain setting using a scouting image radiation dose;
employing a radiation dose model to provide a diagnostic image radiation dose based on the scouting image;
operating the x-ray sensor at a second gain setting, the second gain setting less than the first gain setting; and
capturing a diagnostic image using the provided diagnostic image radiation dose.

11. The computer program product of claim 10, wherein the diagnostic image radiation dose is greater than the scouting image radiation dose.

12. The computer program product of claim 10, wherein the radiation dose model is a machine learning algorithm trained on a collection of training scouting images paired with training diagnostic images, the training diagnostic images selected to provide a desired diagnostic image quality.

13. The computer program product of claim 10, wherein the radiation dose model is an algorithm, the algorithm configured based on an analysis of a collection of sample scouting images paired with sample diagnostic images, the sample diagnostic images selected to provide a desired diagnostic image quality.

14. The computer program product of claim 10, wherein the process further comprises communicating gain settings and radiation dose settings to the x-ray sensor over a communications link and receiving the scouting image and the diagnostic image from the x-ray sensor over a communications link.

15. An x-ray system comprising the computer program product of claim 10.

16. A method for estimating an optimal radiation dose, the method comprising:
operating, by a processor-based system, an x-ray sensor at a first gain setting;
capturing, by the processor-based system, a scouting image at the first gain setting using a scouting image radiation dose;
employing, by the processor-based system, a radiation dose model to provide a diagnostic image radiation dose based on the scouting image;
operating, by the processor-based system, the x-ray sensor at a second gain setting, the second gain setting less than the first gain setting; and
capturing, by the processor-based system, a diagnostic image using the provided diagnostic image radiation dose.

17. The method of claim 16, wherein the diagnostic image radiation dose is greater than the scouting image radiation dose.

18. The method of claim 16, wherein the radiation dose model is a machine learning algorithm trained on a collection of training scouting images paired with training diagnostic images, the training diagnostic images selected to provide a desired diagnostic image quality.

19. The method of claim 16, wherein the radiation dose model is an algorithm, the algorithm configured based on an analysis of a collection of sample scouting images paired with sample diagnostic images, the sample diagnostic images selected to provide a desired diagnostic image quality.

20. The method of claim 16, further comprising communicating gain settings to the x-ray sensor and radiation dose settings to an x-ray emitter over a communications link and receiving the scouting image and the diagnostic image from the x-ray sensor over a communications link.
